(19) United States
(12) Patent Application Publication
(10) Pub. No.: US 2011/0182772 A1
(43) Pub. Date: Jul. 28, 2011

(54) Microbe Reduction and Purification

(76) Inventor: Alton R. Holt, Silsbee, TX (US)

(21) Appl. No.: 12/693,902

(22) Filed: Jan. 26, 2010

(51) Int. Cl. A61L 2/16; B01J 19/12; A61L 9/015

(52) U.S. Cl. 422/29; 422/186; 422/186.3

(57) ABSTRACT

A method and device degrade microbes. In one embodiment, the device includes a purification device for producing hydrogen peroxide gas from air. The air comprises water vapor and oxygen. The purification device includes a body comprising an interior, an air inlet, and a gas outlet. The purification device also includes a catalyst. The catalyst is disposed within the interior. The catalyst comprises titanium dioxide. In addition, the purification device includes a light disposed to emit electromagnetic radiation into the catalyst. Moreover, the purification device includes a fan. The fan is disposed to blow air in a direction. The catalyst is disposed at an angle in relation to the direction.

### CROSS-REFERENCE TO RELATED APPLICATIONS

Not applicable.

### STATEMENT REGARDING FEDERALLY SPONSORED RESEARCH OR DEVELOPMENT

Not applicable.

### BACKGROUND OF THE INVENTION

1. Field of the Invention

This invention relates to the field of antimicrobials and more specifically to the field of hydrogen peroxide gas as an antimicrobial.

2. Background of the Invention

There is an increasing need for disinfection processes against bacteria, viruses, mold, and the like. Conventional disinfection processes involve the application of detergents and liquid sanitizers. Drawbacks to such conventional methods include inefficiencies disinfecting in certain locations such as between walls. Further drawbacks include inefficiencies in the frequency of the disinfection. For instance, such conventional disinfection processes are typically carried out on a daily basis or intermittently during a day.

Developments over such conventional processes include using hydrogen peroxide as a disinfectant. Disinfectant processes using hydrogen peroxide include vaporizing liquid hydrogen peroxide solutions to create a mist of water droplets containing hydrogen peroxide. Drawbacks include that such hydrogen peroxide mist may not be used in occupied spaces because the mist typically contains hundreds to thousands of parts per million of hydrogen peroxide. Further drawbacks include inefficiencies in disinfecting a volume of space because the droplets in the mist precipitate out of the air. Additional drawbacks include that the hydrogen peroxide in the mist is surrounded by water, which may insulate the hydrogen peroxide molecules in the droplets and may prevent the molecules from being drawn to the microbes in the air or on surfaces by electrostatic attraction.

Consequently, there is a need for an improved antimicrobial system for disinfection of surfaces and the air. Additional needs include an improved antimicrobial system using hydrogen peroxide.

### BRIEF SUMMARY OF SOME OF THE PREFERRED EMBODIMENTS

These and other needs in the art are addressed in one embodiment by a purification device for producing hydrogen peroxide gas from air. The air comprises water vapor and oxygen. The purification device includes a body comprising an interior, an air inlet, and a gas outlet. The purification device also includes a catalyst. The catalyst is disposed within the interior. The catalyst comprises titanium dioxide. The purification device also includes a light disposed to emit electromagnetic radiation into the catalyst. In addition, the purification device includes a fan. The fan is disposed to blow air in a direction. The catalyst is disposed at an angle in relation to the direction.

In other embodiments, these and other needs in the art are addressed by a method of degrading microbes. The method includes feeding air to a catalyst. The catalyst comprises titanium dioxide. The air comprises water vapor and oxygen. The method also includes emitting electromagnetic radiation from a light, wherein the electromagnetic radiation contacts the catalyst. The method further includes reacting the air in the presence of the catalyst and the electromagnetic radiation to produce hydrogen peroxide gas. In addition, the method includes degrading the microbes with the hydrogen peroxide gas.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other embodiments for carrying out the same purposes of the present invention. It should also be realized by those skilled in the art that such equivalent embodiments do not depart from the spirit and scope of the invention as set forth in the appended claims.

### BRIEF DESCRIPTION OF THE DRAWINGS

For a detailed description of the preferred embodiments of the invention, reference will now be made to the accompanying drawings in which:

FIG. 1 illustrates a purification device including catalysts and a cover with the cover in an open position;

FIG. 2 illustrates the purification device of FIG. 1 with the cover in a closed position;

FIG. 3 illustrates a side perspective view of the purification device of FIG. 1 showing a meter and a display;

FIG. 4 illustrates a view of the back side of the purification device of FIG. 1 showing air inlet and power inlet;

FIG. 5 illustrates an embodiment of a purification device with a gas outlet on a longitudinal end;

FIG. 6 illustrates an embodiment of a purification device with a plurality of gas outlets; and

FIG. 7 illustrates an alternative embodiment of a purification device.

### DETAILED DESCRIPTION OF THE PREFERRED EMBODIMENTS

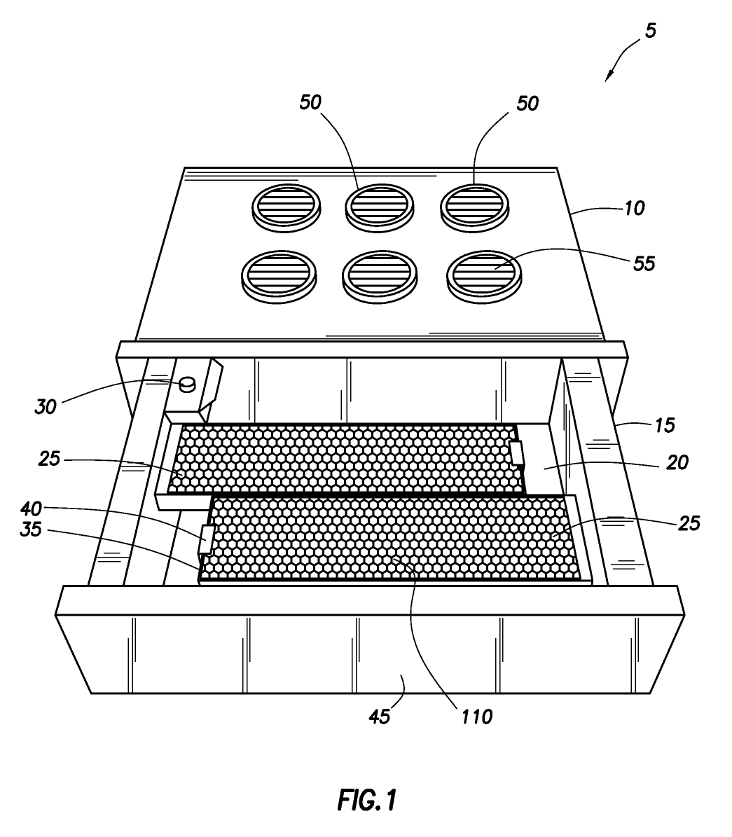

FIG. 1 illustrates an embodiment of a purification device 5 comprising cover 10 and body 15. In an embodiment, purification device 5 creates hydrogen peroxide gas from water vapor and oxygen contained in ambient air. In the embodiment as illustrated in FIG. 1, cover 10 is slidably attached to body 15. In embodiments, cover 10 is slidably attached to allow access to interior 20 of body 15 when cover 10 is in an open position. Cover 10 is slidable in the horizontal and/or vertical direction. In alternative embodiments (not illustrated), cover 10 is not slidable but is connected to body 15 by a hinge or hinges, which allow cover 10 to be moved and allow access to interior 20.

As shown in FIG. 1, purification device 5 comprises catalysts 25. Catalysts 25 are disposed in interior 20. The embodiment of purification device 5 shown in FIG. 1 has two catalysts 25. However, purification device 5 is not limited to two catalysts 25 but may have one catalyst 25 or more than two catalysts 25. Catalyst 25 comprises titanium dioxide. In other embodiments, catalyst 25 comprises titanium dioxide and metallic additives. Any metallic additives suitable for improving the reaction to produce the hydrogen peroxide may be used. In an embodiment, the metallic additives include copper, silver, rhodium, or any combinations thereof. Catalyst 25 may have any suitable configuration for use in purification device 5. In embodiments, catalyst 25 comprises a configuration of a plurality of cells. In an embodiment as illustrated in FIG. 1, catalyst 25 comprises a configuration of a plurality of hexagonal, walled cells (i.e., honeycomb shape configuration). Without being limited by theory, the hexagonal, walled cell configuration facilitates the reaction to produce the hydrogen peroxide because it provides an increased surface area for the reaction. The embodiment of catalyst 25 shown in FIG. 1 has a rectangular shape but is to be understood that catalyst 25 is not limited to a rectangular shape but in alternative embodiments may have any other suitable shape such as a square shape, triangular shape, and the like. In some embodiments (not illustrated), catalyst 25 is disposed at an angle in relation to the direction at which the fans blow the air. In embodiments, catalyst 25 is disposed at angle in relation to the direction at which the fans blow the air of between about 15 degrees and about 75 degrees, and alternatively at about 45 degrees. Without being limited by theory, disposing catalyst 25 at an angle to the direction at which the fans blow the air increases the surface area available for the reaction to produce the hydrogen peroxide. For instance, as light and air pass through catalyst 25, the catalyst 25 disposed at an angle increases the amount of contact of the light and air with the surface of catalyst 25. In some embodiments as illustrated in FIG. 1, a catalyst container 35 may be disposed about the outer side edges of catalyst 25. Further embodiments as illustrated may also include catalyst container 35 having stop 40. Catalyst container 35 provides support to catalyst 25 and also prevents movement of catalyst 25 within interior 20. Stop 40 is an angled portion of catalyst container 35 that in some embodiments is disposed parallel to catalyst 25. Stop 40 prevents unwanted lateral movement of catalyst 25 and maintains catalyst 25 within catalyst container 35 during typical operation of purification device 5. In an embodiment, catalyst container 35 is secured to body 15. Catalyst container 35 may be secured to body 15 by any suitable means.

Catalyst 25 includes a light (not illustrated) disposed inside the catalyst body 110. The light is a non-ozone producing ultraviolet light. In embodiments, the light is a crystal ultraviolet light. Without limitation, commercial examples of non-ozone producing ultraviolet lights include the non ozone bulb provided by LightTech. Catalyst 25 includes one light. In alternative embodiments, catalyst 25 includes more than one light. The light is disposed to emit electromagnetic radiation into catalyst 25. For instance, the light emits electromagnetic radiation into the hexagonal, walled cells of catalyst 25 with the electromagnetic radiation contacting the surface of the cells. In alternative embodiments (not illustrated), the light is not disposed within catalyst 25 but is instead located out of catalyst 25 and disposed to emit electromagnetic radiation that contacts catalyst 25.

As shown in FIG. 1, embodiments of purification device 5 also include switch 30. Switch 30 may be a safety switch. For instance, switch 30 may shut off operation of purification device 5 in an instance in which cover 10 is actuated and opens interior 20 during operation of purification device 5. Any suitable safety switch for shutting off operation of purification device 5 may be used.

FIG. 1 shows an embodiment of purification device 5 having base 45. Base 45 provides support for purification device 5.

FIG. 1 also shows cover 10 having gas outlets 50. FIG. 1 shows purification device 5 having six gas outlets 50, but it is to be understood that purification device 5 is not limited to six gas outlets as purification device 5 may have any desirable number of gas outlets. The produced hydrogen peroxide gas exits purification device 5 through gas outlets 50. Gas outlets 50 may have any suitable configuration for allowing the produced hydrogen peroxide gas to exit purification device 5. In some embodiments as illustrated in FIG. 1, gas outlets 50 have gas outlet covers 55. In an embodiment, gas outlet covers 55 comprise louvers. Without limitation, the louvers allow the produced hydrogen peroxide gas to exit purification device 5, but provide protection for the eyes of people in proximity to purification device 5 from damage from the electromagnetic radiation produced by the light in catalyst 25. In embodiments, the louvers are adjustable to direct the flow of hydrogen peroxide gas.

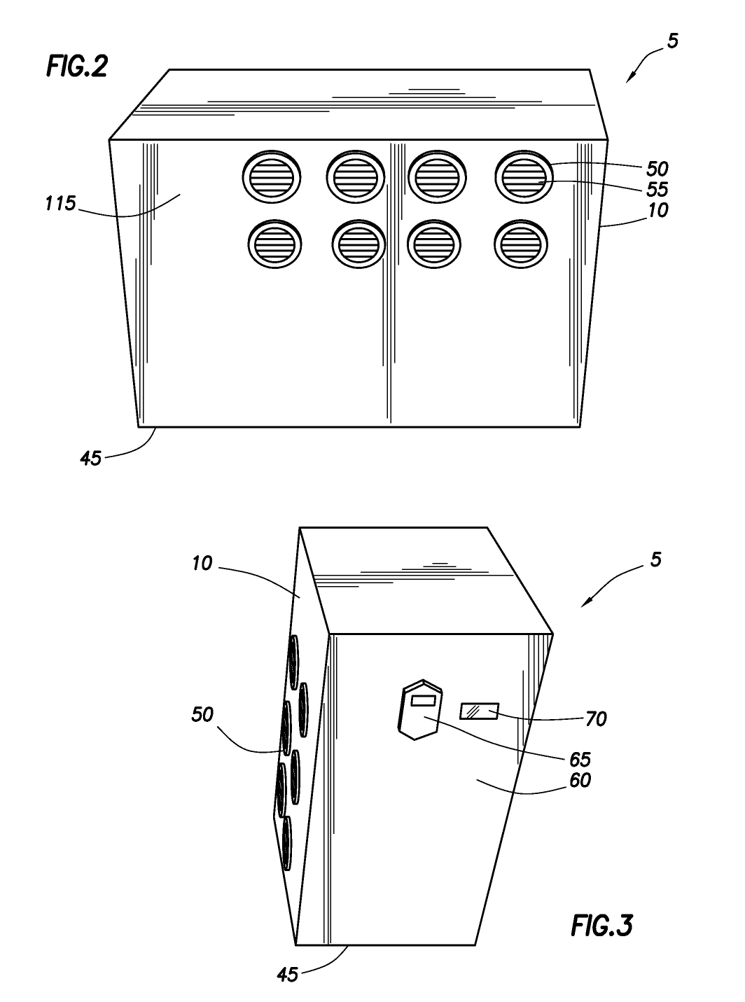

FIG. 2 illustrates a front perspective view of an embodiment of purification device 5 with cover 10 in a closed position. In an embodiment as shown, gas outlets 50 are in an upper portion 115 of purification device 5. Without limitation, gas outlets 50 are disposed in upper portion 115 to reduce any obstructions to the flow of the produced hydrogen peroxide gas out of purification device 5. However, it is to be understood that gas outlets 50 are not limited to disposition in upper portion 115, but in alternative embodiments (not illustrated) are disposed at any location on cover 10. In some alternative embodiments (not illustrated), gas outlets 50 are disposed on other sides of purification device 5 besides or in addition to cover 10.

FIG. 3 illustrates a side perspective view of an embodiment of purification device 5 having a meter 65 and display 70 on side 60. Meter 65 may be a meter that measures data such as operating time of purification device 5. Display 70 may display information such as the operating time, temperature, and the like. It is to be understood that meter 65 and display 70 are not limited to disposition on side 60 but in alternative embodiments may be disposed at any desirable location on purification device 5.

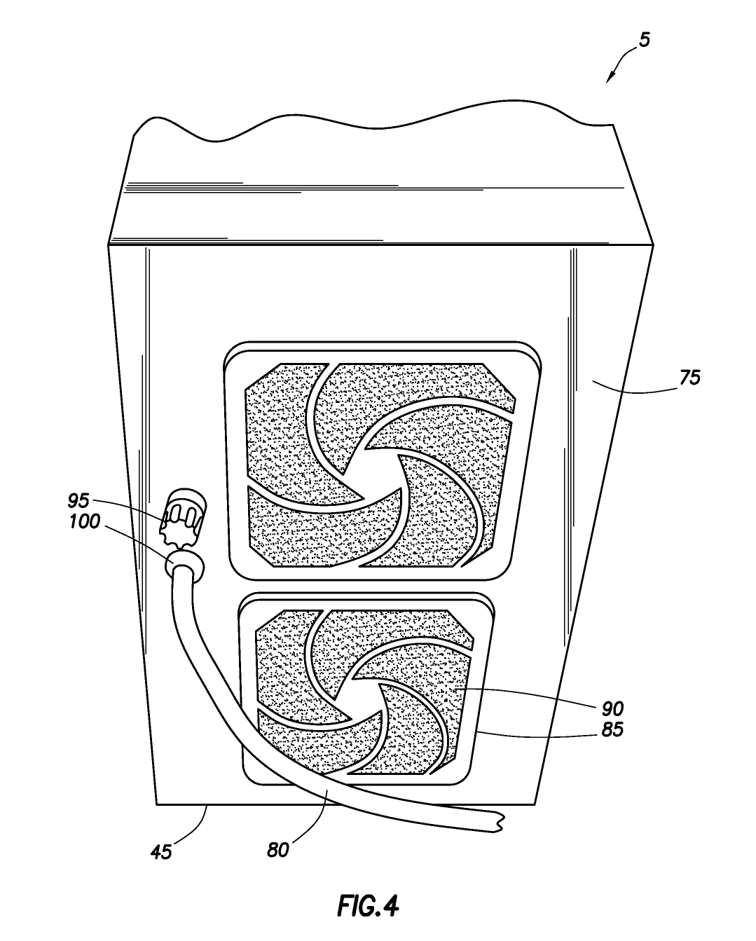

FIG. 4 illustrates an embodiment of purification device 5 in which back side 75 of purification device 5 has air inlets 85. In embodiments, purification device 5 has fans (not illustrated) disposed in interior 20. The fans take air from outside purification device 5 and provide such air to catalyst 25. In embodiments, the fans are disposed between catalyst 25 and back side 75. Purification device 5 may have any desirable number of fans. In some embodiments, purification device 5 has one fan for each catalyst 25. The fans may be any fan of a power suitable for providing ambient air through catalyst 25 and then providing sufficient air pressure in purification device 5 to force the produced hydrogen peroxide out of purification device 5. Air inlets 85 allow air to pass therethrough to interior 20. In some embodiments as illustrated, inlet covers 90 are disposed over air inlets 85. Without limitation, inlet covers 90 prevent unwanted obstructions from being pulled into interior 20. In some embodiments as illustrated, inlet covers 90 also reduce the amount of dust particles and the like from entering interior 20. As further illustrated, in some embodiments, back side 75 has fuse 95 and power inlet 100. Fuse 95 is any suitable fuse for use with purification device 5. Power inlet 100 is the power supply to purification device 5. The power may be supplied to power inlet 100 by any suitable means. In an embodiment as illustrated in FIG. 4, the power is supplied by power cord 80. It is to be understood that fuse 95 and power inlet 100 may in alternative embodiments (not illustrated) be disposed at any desired location on any side of purification device 5.

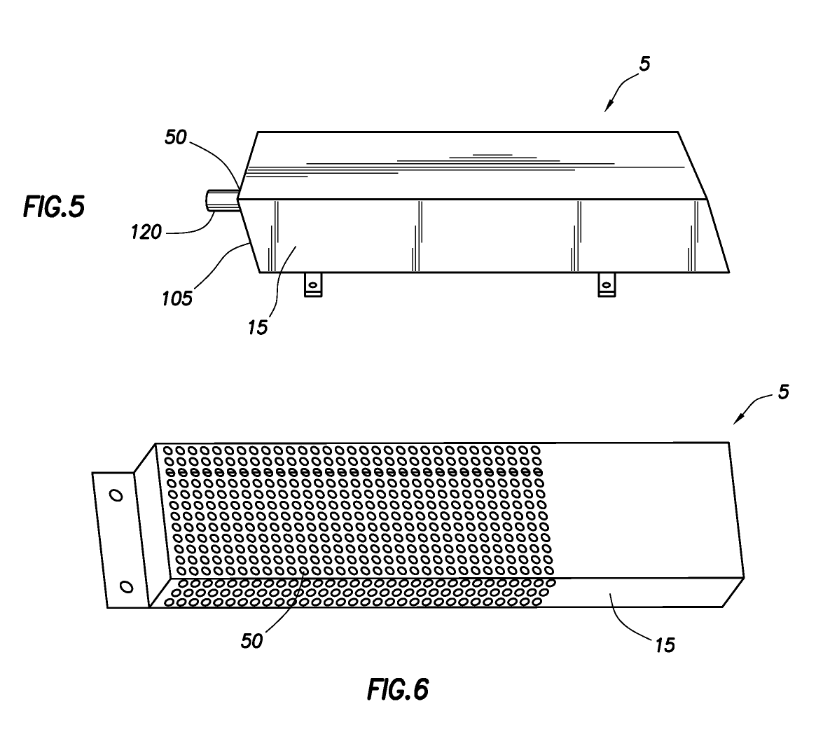

FIG. 5 illustrates a side perspective view of an embodiment of purification device 5 in which gas outlet 50 is disposed on a longitudinal end 105 of purification device 5. In such an embodiment, purification device 5 includes a gas outlet tube 120 secured to gas outlet 50. Gas outlet tube 120 may be secured to gas outlet 50 by any suitable means. In embodiments, gas outlet tube 120 allows the direction of the produced hydrogen peroxide gas exiting purification device 5 to be controlled through gas outlet tube 120.

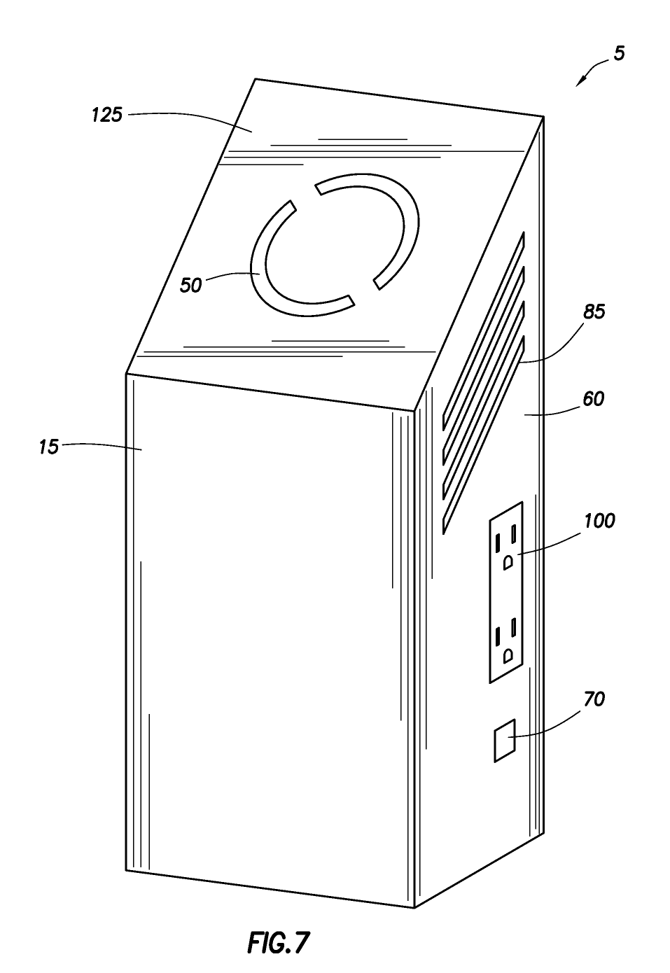

FIG. 6 illustrates an embodiment of a purification device 5 in which body 15 has a plurality of gas outlets 50. As shown, gas outlets 50 do not have gas outlet covers 55. In an embodiment, air is directed across gas outlets 50, contacting catalyst 25 and producing the hydrogen peroxide gas. In embodiments, the embodiment of purification device 5 is disposed in an air conditioner system.

FIG. 7 illustrates an embodiment of a purification device 5 with power inlet 100, display 70, and air inlets 85 on side 60. In such an embodiment, gas outlet 50 is not disposed on cover 10 but is instead disposed on a top portion 125 of body 15.

In an embodiment as illustrated in FIGS. 1-7, operation of purification device 5 includes placement of purification device 5 in a room, facility or the like or near any other location in which it is desired to control microbial contamination. Purification device 5 is activated (i.e., by operation of switch 30), which activates the fans and the lights. The fans pull ambient air through air inlets 85 into interior 20. The ambient air is then blown by the fans to catalyst 25 with the air passing through catalyst 25. It is to be understood that the ambient air has a moisture content and is comprised of water vapor and oxygen. Catalyst 25 and the moisture in the ambient air (i.e., the water vapor and oxygen) are exposed to the electromagnetic radiation from the lights. A reaction between the titanium dioxide, the moisture in the ambient air, and the electromagnetic radiation produce the hydrogen peroxide gas. In an embodiment, the reaction is a photo-catalytic reaction. For instance, in embodiments, moisture from the ambient air contacts catalyst 25 as it flows through catalyst 25. The electromagnetic radiation from the light contacts the various surfaces of catalyst 25 and reacts with the moisture against the titanium dioxide to produce the hydrogen peroxide gas. In an embodiment as illustrated in which catalyst 25 has a hexagonal, walled cell configuration, such configuration facilitates the reaction because it increases the contact of the electromagnetic radiation and the ambient air with the titanium dioxide as the electromagnetic radiation and the ambient air pass through catalyst 25 and contact the surfaces of the various walled cells. In embodiments in which catalyst 25 is angled in relation to the direction at which the fans are blowing the air, the contact of the electromagnetic radiation and the ambient air with the titanium dioxide is increased, which further facilitates the reaction. For instance, by changing the angle the surface reaction is increased, which may cause increased contact (i.e., multiple contacts) of electrons with catalyst 25. The reaction in purification device 5 to produce the hydrogen peroxide gas does not produce ozone. In an embodiment, the reaction produces less than about 0.05 parts per million (ppm) of the hydrogen peroxide gas from the air, alternatively less than about 0.02 ppm, and alternatively from about 0.02 ppm to about 0.05 ppm.

As further shown in the embodiments illustrated in FIGS. 1-7, air pressure from the fans directs the produced hydrogen peroxide gas away from catalyst 25 and out of purification device 5 through gas outlets 50. In an embodiment, operation of purification device 5 continuously produces hydrogen peroxide gas while in operation. In other embodiments, purification device 5 may be turned off by manual operation of switch 30.

Without being limited by theory, the produced hydrogen peroxide gas has both positive and negative charges. With such charges, the hydrogen peroxide gas is drawn to microbes by electrostatic attraction. For instance, the hydrogen peroxide gas is drawn to the positive and negative charges of the surface of the microbes. The hydrogen peroxide gas then chemically degrades the microbes, which may be degraded cell by cell. In embodiments in which the microbes are attached to structures such as a wall, the hydrogen peroxide gas degrades the microbes down to the point of attachment. In some instances, the microbes release from the surface and may be removed. In embodiments, the microbes may be removed without removing structurally sound material. The hydrogen peroxide gas also diffuses into porous material (i.e., anywhere that air flows) such as porous walls and cloth, which allows degradation of the microbes behind the walls or in the cloth.

It is to be understood that purification device 5 is not limited to ambient air but in alternatives may use other suitable gases or vapors that contain water vapor and oxygen and that are suitable for producing hydrogen peroxide. For instance, a mixture of water vapor and oxygen may be provided to purification device 5 (i.e., by a tank).

The microbes may include any type of microbe. In an embodiment, the microbes comprise fungi, mold, viruses, bacteria, or any combinations thereof.

To further illustrate various illustrative embodiments of the present invention, the following examples are provided.

### Example 1

The effect of hydrogen peroxide produced from a purification device on the inactivation of Influenza A H1N1 (ATCC # VR-333) was evaluated. The virus culture was maintained on an ATCC complete growth medium and minimum essential medium (ATCC, Manassas, Va., USA) with 2 μM L-glutamine and Earle's BSS adjusted to contain 1.5 g/L sodium bicarbonate, 0.1 μM non-essential amino acids, and 1.0 μM sodium pyruvate. 90% fetal bovine serum, 10% cultured in Trypticase Soy Agar was added. Sodium bicarbonate, non-essential amino acids, and a combination of sodium pyruvate and fetal bovine serum, in aerobic growth conditions at 37.0° C. and Influenza A at 33-35° C. Cells from both of the above (approx. 1×107 CFU/ml) from a 24 hour static culture were incubated at 37.0° C., and Influenza A at 33-35° C. were used to inoculate various 5 cm×3 cm stainless steel coupons. The inoculum suspensions were enumerated by surface plating in duplicate samples on TSA after serial dilution in 0.1% peptone solution. The plates were incubated for 24 hour at 37.0° C.

A 100 μl droplet from the initial inoculum suspension of each of the bacteria/viruses was used to inoculate the external surface (6.3 cm×1.8 cm) on #8 stainless steel coupons. This resulted in a final inoculum level of approximately 7.0-log CFU/5 g sample. The inoculated samples were air dried for 1 hour at 22.0° C. prior to treatment with the no-ozone producing cell. The 1 hour drying allowed the inoculated cells to attach to the surface host and minimize the growth of inoculated cells during drying. Four stainless steel coupons were used for each sampling time.

A biocontainment chamber (BL 2 Enhanced) was equipped with a purification device and allowed to equilibrate for a period of two hours prior to placement of 12 inoculated coupons inside the chamber. The purification device used was a 14 inch cell. The cell was placed on the outside of the box, and the gases were blown into the container. The effect of the no-ozone producing cell treatment was measured at 0, 2, 4, 6, 8, 12 and 24 hours. A control study was conducted in the same chamber without the presence of the purification device. Temperature, relative humidity, and ambient ozone levels and hydrogen peroxide levels were monitored in the chamber.

After treatment, each of the 5 cm×3 cm coupons were transferred into a 400 ml stomacher bag (Fisher Scientific Inc., PA., USA) combined with 50 ml sterile 0.1% peptone solution, and then blended with a AES Easy Mix Stomacher (AES Laboratories, Princeton, N.J., USA) for 2 minutes at normal speed. Wash fluid was serially diluted, followed by surface plating for enumeration. A centrifugation method was used to recover low populations of injured viruses. The centrifugation method (Mossel and others 1991) was modified and used to concentrate the virus populations in the wash fluid so that less than 250 CFU/ml of the virus could be enumerated by the surface plating.

TABLE 1
Average recoveries (Log CFU/cm²) of Influenza A H1N1on
inoculated stainless steel coupons treated using the purification
device for periods of 0, 2, 4, 6, 8, 12 and 24 Hours.

| | | Influenza A H1N1 | |
|---|---|---|---|
| | | Treated with | |
| | | No-Ozone | Influenza A |
| | | producing Cell | H1N1 Control |
| Sample | | Log CFU/cm² | Log CFU/cm² |
| Initial-0 | Time | 7.1 | 6.9 |
| 2 | Hours | 4.2 | 6.6 |
| 4 | Hours | 2.4 | 6.7 |
| 6 | Hours | BDL | 6.6 |
| 8 | Hours | BDL | 6.2 |
| 12 | Hours | BDL | 6.2 |
| 24 | Hours | BDL | 6.3 |

Table 1 summarizes the results of the example, which demonstrated the effectiveness of the purification device for the inactivation of Influenza A—H1N1. After 6 hours of treatment, levels of the H1N1 virus on inoculated stainless steel coupons were below the detection limit. No recovery was observed at 8, 12, or 24 hours.

The ambient ozone levels in the chamber containing the purification device were measured at 0.0055 ppm. Ozone levels in the control chamber were measured at 0.0050 ppm (no significant difference). Levels of vaporized hydrogen peroxide in the chamber equipped with the purification device ranged from 0.05-0.08 ppm. No vaporized H₂O₂ was measured in the control chamber. The relative humidity ranged from 47-59%, and the temperature from 70-72° F. in both the control and treated chambers.

The results indicated that the purification device was effective at inactivating Influenza A H1N1 virus on inoculated stainless coupons under the conditions of these tests.

Although the present invention and its advantages have been described in detail, it should be understood that various changes, substitutions and alterations may be made herein without departing from the spirit and scope of the invention as defined by the appended claims.

What is claimed is:

1. A purification device for producing hydrogen peroxide gas from air, wherein the air comprises water vapor and oxygen, comprising:
a body comprising an interior, an air inlet, and a gas outlet;
a catalyst, wherein the catalyst is disposed within the interior, and wherein the catalyst comprises titanium dioxide;
a light disposed to emit electromagnetic radiation into the catalyst; and
a fan, wherein the fan is disposed to blow air in a direction, and wherein the catalyst is disposed at an angle in relation to the direction.

2. The purification device of claim 1, wherein the catalyst comprises a metallic additive.

3. The purification device of claim 2, wherein the metallic additive comprises copper, silver, rhodium, or any combinations thereof.

4. The purification device of claim 1, wherein the catalyst comprises a plurality of hexagonal, walled cells.

5. The purification device of claim 1, wherein the angle is between about 15 degrees and about 75 degrees.

6. The purification device of claim 1, wherein the light comprises a non-ozone producing ultraviolet light.

7. The purification device of claim 1, wherein the light is disposed within the catalyst.

8. The purification device of claim 1, wherein the gas outlet is disposed on an opposing side of the air purification device from the air inlet.

9. The purification device of claim 1, wherein the catalyst is disposed to provide a reaction surface by which the air reacts when exposed to the catalyst and the electromagnetic radiation.

10. The purification device of claim 1, wherein producing the hydrogen peroxide gas does not produce ozone.

11. A method of degrading microbes, comprising:
(A) feeding air to a catalyst, wherein the catalyst comprises titanium dioxide, and wherein the air comprises water vapor and oxygen;
(B) emitting electromagnetic radiation from a light, wherein the electromagnetic radiation contacts the catalyst;
(C) reacting the air in the presence of the catalyst and the electromagnetic radiation to produce hydrogen peroxide gas; and
(D) degrading the microbes with the hydrogen peroxide gas.

12. The method of claim 11, wherein the catalyst further comprises copper, silver, rhodium, or any combinations thereof.

13. The method of claim 11, wherein the catalyst comprises a plurality of hexagonal, walled cells.

14. The method of claim 11, further comprising feeding the air to the catalyst with the catalyst disposed at an angle.

15. The method of claim 14, wherein the angle is between about 15 degrees and about 75 degrees.

16. The method of claim 11, wherein the light comprises a non-ozone producing ultraviolet light.

17. The method of claim 11, wherein the light is disposed within the catalyst.

18. The method of claim 11, wherein the production of the hydrogen peroxide gas does not produce ozone.

19. The method of claim 11, wherein the air comprises ambient air.

20. The method of claim 11, wherein the air flows through the catalyst.
